(19) United States
(12) Patent Application Publication
(10) Pub. No.: US 2005/0048468 A1
(43) Pub. Date: Mar. 3, 2005

(54) Multiconstituent liquid lgG and IgM calibrators

(75) Inventors: Peilin Chen, Cupertino, CA (US); Hao Yu, Pacheco, CA (US); Lawrence Blecka, Walnut Creek, CA (US); Steve Binder, Berkeley, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 10/650,595

(22) Filed: Aug. 27, 2003

(51) Int. Cl. C12Q 1/70; A61K 39/42

(52) U.S. Cl. 435/5; 424/159.1

(57) ABSTRACT

A serum in which are dissolved at least two heterologous antibodies, which are independently IgG or IgM and which specifically bind to different antigens, serves as a calibrator for multi-analyte immunoassays.

### BACKGROUND OF THE INVENTION

Diagnosis of infectious diseases often relies on serological assays for detecting pathogen-specific immunoglobulins in a biological sample (e.g., a serum sample from a patient). It is necessary to include in such assays positive controls or calibrators to indicate the presence as well as the level of antibodies against antigens of pathogenic organisms, such as viruses and bacteria.

It is well known that one of the first immune responses to an infectious agent is the production of IgM antibodies. This IgM response lasts for a short period of time (e.g., a few weeks to a few months) due to the half life of IgM antibodies. Following the IgM immune response is the IgG response, which lasts longer, typically several years. The presence of either IgM or IgG specifically binding to particular pathogens in a patient's serum can indicate exposure to such pathogens at different times.

Sera obtained from subjects known to have prior exposure to certain infectious organisms have traditionally been used as positive calibrators once the presence of antibodies against these pathogens is confirmed. The main drawbacks of the use of calibrators from natural sources are: first, the antibodies present in naturally occurring sera are few in number and are specifically reactive to only the most frequently tested pathogens, naturally occurring sera are thus unsuitable as calibrators in a multi-analyte assay system; second, the levels of antibodies naturally present in a serum may differ significantly for different pathogens, which is another reason why the serum is an undesirable calibrator; third, although multiple sera may be combined to produce a mixture that contains of a pool of antibodies specific for a sufficient number of different antigens, the concentration of the antibodies in such a serum mixture tends to be too diluted for the mixture to properly serve as a calibrator. Thus, there exists a need for a multi-constituent liquid calibrator that contains antibodies against a sufficient number of different antigens, where the concentrations of antibodies against each antigen are comparable and suitable for calibration purposes. The present invention addresses this and related needs.

### SUMMARY OF THE INVENTION

The present invention resides in multiconstituent liquid IgG and IgM calibrators that can be used as positive controls in multi-analyte immunoassays. One aspect of the present invention is a composition that includes a serum and a plurality of heterologous antibodies dissolved in the serum. These antibodies are independently IgG or IgM and specifically bind to different antigens. The composition contains at least two, preferably at least three, four, five, six, seven, eight, nine, ten, eleven, twelve, thirteen, fourteen, fifteen, sixteen, seventeen, or even more heterologous antibodies.

In some embodiments, the composition includes antibodies specifically reactive to different antigens derived from one or more organisms independently selected from the group consisting of Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Epstein-Barr Virus (EBV), Varicella Zoster Virus, Borrelia burgdorferi, Treponema pallidum, Helicobacter pylori, and Mycoplasma pneumoniae. Particularly, the composition may include one or more antibodies specifically reactive to different antigens derived from EBV: EBV Viral Capsid Antigen (EBV-VCA), EBV Nuclear Antigen type-1 (EBV-NA1), EBV Early Antigen Diffused (EBV-EAD), and EBV Early Antigen Restricted (EBV-EAR).

In some preferred embodiments, the composition includes antibodies specifically reactive to antigens derived from Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

In other preferred embodiments, the composition includes antibodies specifically reactive to antigens derived from Rubella virus, Mumps Virus, and Measles Virus.

In other preferred embodiments, the composition includes antibodies specifically reactive to antigens derived from Toxoplasma gondii, Rubella virus, Cytomegalovirus (CMV), Herpes Simplex Virus type-1 (HSV-1), and Herpes Simplex Virus type-2 (HSV-2).

In other preferred embodiments, the composition includes antibodies specifically reactive to antigens derived from Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Varicella Zoster Virus, Treponema pallidum, Helicobacter pylori, Epstein-Barr Virus Viral Capsid Antigen (EBV-VCA), Epstein-Barr Virus Nuclear Antigen type-1 (EBV-NAI), and Epstein-Barr Virus Early Antigen Diffused (EBV-EAD).

In yet other preferred embodiments, the composition includes antigens specifically reactive to antigens derived from Toxoplasma gondii, Rubella virus, Cytomegalovirus (CMV), Herpes Simplex Virus type-i (HSV-1), Herpes Simplex Virus type-2 (HSV-2), Mumps Virus, Measles Virus, Varicella Zoster Virus, Borrelia burgdorferi, Treponema pallidum, Helicobacter pylori, and Mycoplasma pneumoniae, Epstein-Barr Virus Viral Capsid Antigen (EBV-VCA), Epstein-Barr Virus Nuclear Antigen type-I (EBV-NA1), Epstein-Barr Virus Early Antigen Diffused (EBV-EAD), and Epstein-Barr Virus Early Antigen Restricted (EBV-EAR).

Another aspect of the present invention is a method for the manufacture of a composition including a serum and a plurality of heterologous antibodies dissolved in the serum, where these heterologous antibodies are independently IgG or IgM and specifically bind to different antigens. Such method includes the following steps: first, a first antibody is purified and quantified; second, the first antibody is dissolved in a serum containing at least one antibody that is heterologous to the first antibody.

A further aspect of the present invention is a method for the manufacture of a composition for use in detecting the presence and amount of IgG or IgM antibodies in a biological sample. The method includes the following steps: first, a plurality of heterologous antibodies specifically reactive to different antigens are purified and quantified; second, these heterologous antibodies are dissolved in a serum that already contains an antibody heterologous to the antibodies purified and quantified in the first step and specifically reactive to an antigen different from those to the heterologous antibodies.

The composition contains at least two, preferably at least three, four, five, six, seven, eight, nine, ten, eleven, twelve, thirteen, fourteen, fifteen, sixteen, seventeen, or even more heterologous antibodies. In some embodiments, the composition includes antibodies specifically reactive to different antigens derived from Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Epstein-Barr Virus (EBV), Varicella Zoster Virus, Borrelia burgdorferi, Treponema pallidum, Helicobacter pylori, and Mycoplasma pneumoniae. Particularly, the composition may include antibodies specifically reactive to different antigens derived from EBV: EBV Viral Capsid Antigen (EBV-VCA), EBV Nuclear Antigen type-1 (EBV-NA1), EBV Early Antigen Diffused (EBV-EAD), and EBV Early Antigen Restricted (EBV-EAR).

In some preferred embodiments, the composition includes antibodies specifically reactive to antigens derived from Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

In other preferred embodiments, the composition includes antibodies specifically reactive to antigens derived from Rubella virus, Mumps Virus, and Measles Virus.

In other preferred embodiments, the composition includes antibodies specifically reactive to antigens derived from Toxoplasma gondii, Rubella virus, Cytomegalovirus (CMV), Herpes Simplex Virus type-1 (HSV-1), and Herpes Simplex Virus type-2 (HSV-2).

In other preferred embodiments, the composition includes antibodies specifically reactive to antigens derived from Toxoplasma gondii, Rubella virus, Cytomegalovirus (CMV), Herpes Simplex Virus type-I (HSV-1), Herpes Simplex Virus type-2 (HSV-2), Mumps Virus, Measles Virus, Varicella Zoster Virus, Treponema pallidum, Helicobacter pylori, Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

In yet other preferred embodiments, the composition includes antigens specifically reactive to antigens derived from Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Varicella Zoster Virus, Borrelia burgdorferi, Treponema pallidum, Helicobacter pylori, and Mycoplasma pneumoniae, Epstein-Barr Virus Viral Capsid Antigen (EBV-VCA), Epstein-Barr Virus Nuclear Antigen type-1 (EBV-NA1), Epstein-Barr Virus Early Antigen Diffused (EBV-EAD), and Epstein-Barr Virus Early Antigen Restricted (EBV-EAR).

A yet further aspect of the invention is a method for analyzing a biological sample to detect the presence and amount of IgG or IgM antibodies to predetermined different antigens. The method includes the following steps: first, contacting the biological sample with a plurality of predetermined different antigens under conditions sufficient to allow the formation of antigen/antibody complexes between the antigens and any antibodies present in the sample that specifically bind to the antigens; second, detecting any antigen/antibody complexes formed in the first step; third, comparing the result from the second step to a result obtained from a similar procedure where a control composition is used, in order to identify any of the antibodies present in the sample and to quantify the levels of the antibodies. The control composition includes a serum and a plurality of heterologous antibodies dissolved in the serum, which are independently IgG or IgM and specifically bind to the predetermined different antigens.

The composition contains at least two, preferably at least three, four, five, six, seven, eight, nine, ten, eleven, twelve, thirteen, fourteen, fifteen, sixteen, seventeen, or even more heterologous antibodies. In some embodiments, the composition includes antibodies specifically reactive to different antigens derived from Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Epstein-Barr Virus (EBV), Varicella Zoster Virus, Borrelia burgdorferi, Treponema pallidum, Helicobacter pylori, and Mycoplasma pneumoniae. Particularly, the composition may include antibodies specifically reactive to different antigens derived from EBV: EBV Viral Capsid Antigen (EBV-VCA), EBV Nuclear Antigen type-1 (EBV-NA1), EBV Early Antigen Diffused (EBV-EAD), and EBV Early Antigen Restricted (EBV-EAR).

In some preferred embodiments, the composition includes antibodies specifically reactive to antigens derived from Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

In other preferred embodiments, the composition includes antibodies specifically reactive to antigens derived from Rubella virus, Mumps Virus, and Measles Virus.

In other preferred embodiments, the composition includes antibodies specifically reactive to antigens derived from Toxoplasma gondii, Rubella virus, Cytomegalovirus (CMV), Herpes Simplex Virus type-1 (HSV-1), and Herpes Simplex Virus type-2 (HSV-2).

In other preferred embodiments, the composition includes antibodies specifically reactive to antigens derived from Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Varicella Zoster Virus, Treponema pallidum, Helicobacter pylori, Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

In yet other preferred embodiments, the composition includes antigens specifically reactive to antigens derived from Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Varicella Zoster Virus, Borrelia burgdorferi, Treponema pallidum, Helicobacter pylori, and Mycoplasma pneumoniae, Epstein-Barr Virus Viral Capsid Antigen (EBV-VCA), Epstein-Barr Virus Nuclear Antigen type-1 (EBV-NA1), Epstein-Barr Virus Early Antigen Diffused (EBV-EAD), and Epstein-Barr Virus Early Antigen Restricted (EBV-EAR).

In some preferred embodiments, flow cytometry is used to practice the claimed method for detecting in a biological sample any IgG or IgM antibodies specific to predetermined different antigens.

### Definitions

An “antibody” refers to a glycoprotein of the immunoglobulin family or a polypeptide comprising fragments of an immunoglobulin that is capable of noncovalently, reversibly, and in a specific manner binding a corresponding antigen. The typical antibody structural unit is a tetramer. Each tetramer is composed of two identical pairs of polypeptide chains, each pair having one “light” (about 25 kD) and one “heavy” chain (about 50-70 kD), connected through a disulfide bond. The recognized immunoglobulin genes include the κ, λ, α, γ, δ, ε, and μ constant region genes, as well as the myriad immunoglobulin variable region genes. Light chains are classified as either κ or λ Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (V_{L}) and variable heavy chain (V_{H}) refer to these regions of light and heavy chains respectively.

The term antibody, as used herein, includes both monoclonal and polyclonal antibodies, and encompasses antibodies raised in vivo, e.g., produced by an animal upon immunization by an antigen, and antibodies generated in vitro, e.g., generated by hybridomas. As used in this application, antibodies that specifically recognize the same antigen, e.g., a pathogenic organism, are regarded as “one antibody,” regardless of whether they actually bind to the same or to separate antigenic epitopes of the antigen.

For preparation of monoclonal or polyclonal antibodies, any technique known in the art can be used (see, e.g., Kohler & Milstein, Nature 256:495-497, 1975; Kozbor et al., Immunology Today 4:72, 1983; Cole et al., Monoclonal Antibodies and Cancer Therapy, pp. 77-96. Alan R. Liss, Inc., 1985). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., supra; Marks et al., Biotechnology, 10:779-783, 1992).

The term “serum” as used herein generally refers to a preparation of blood that is essentially free of cells. Thus, in the present application, this term encompasses both serum and plasma in their conventional definitions, unless otherwise indicated.

The term “plurality” as used herein means more than one. A “plurality” of antibodies preferably refers to at least five, more preferably at least ten, and most preferably at least fifteen antibodies in the claimed composition. This term does not indicate any particular order in which the antibodies may be dissolved in the serum of the composition.

The term “heterologous” as used herein describes the relationship among some antibodies present in the claimed composition. An antibody is heterologous to another when the two antibodies do not naturally occur in the same source, i.e., in a blood preparation (such as a serum as conventionally defined) from the same subject. Further, any antibody produced in a process where manipulation of nucleic acids encoding the antibody is required, such as a recombinantly produced antibody, a chimeric antibody, or a fully human monoclonal antibody produced by a transgenic animal, is considered “heterologous” in this application to any other antibodies. Moreover, not all antibodies in a multiconstituent IgG and IgM liquid calibrator of the present invention need be heterologous to each other.

The term “independently” when used herein to describe antibodies as either IgG or IgM, refers to the fact that the antibodies present in a claimed multiconstituent liquid calibrator are either all IgG, all IgM, or a mixture of IgG and IgM at any number and that any one antibody can be either IgG or IgM with no correlation to any other antibody's being IgG or IgM.

The term “different” when used herein to modify antigens refers to antigens that are derived from different organisms, except when in reference to the Epstein-Barr Virus (EBV). “Different antigens” may thus be distinct antigens derived from the same organism—EBV. In other words, while two antigens derived from two different organisms, such as two different viruses, e.g., Herpes Simplex Virus type-1 (HSV-1) and Herpes Simples Virus type-2 (HSV-2), are “different antigens”, two distinct antigens derived from the same organism, e.g., HSV-1, are not regarded as “different antigens” in the present application. As an exception, however, distinct EBV antigens, such as Epstein-Barr Virus Viral Capsid Antigen (EBV-VCA), Epstein-Barr Virus Nuclear Antigen type-1 (EBV-NA1), Epstein-Barr Virus Early Antigen Diffused (EBV-EAD), and Epstein-Barr Virus Early Antigen Restricted (EBV-EAR), are considered “different antigens” in this application. Accordingly, “different” antibodies in this application are defined as antibodies specifically reactive to “different” antigens and the number of “different” antibodies present in a claimed liquid IgG and IgM calibrator are the number of “different” antigens they specifically recognize.

The term “specifically bind” as used herein to describe the interaction between an antigen and an antibody refers to the fact that detection of any antibody bound to a particular antigen is determinative of the presence of the antibody against the antigen, often in a heterogeneous population of other antibodies and proteins. Under designated immunoassay conditions, a detectable signal is designated as one that is at least twice the background signal. Thus, a specific antigen-antibody binding should yield a signal at least two times the background and more typically more than 10 to 100 times the background.

The term “biological sample” refers to sections of tissues such as biopsy and autopsy samples, and frozen sections taken for histological purposes. Such samples may include whole blood, serum (as conventionally defined), plasma (as conventionally defined), cerebrospinal fluid, sputum, tissue, cultured cells, e.g., primary cultures, explants, transformed cells, stool, urine, vesicle fluid, mucus, and other bodily secretion or tissue that could be sampled with a swab device. A biological sample is typically obtained from a subject, e.g., an animal or a human, that may have been infected with one or more infectious organisms included in a predetermined list of organisms to be tested for.

### DETAILED DESCRIPTION OF THE INVENTION

I. INTRODUCTION

The present invention resides in multiconstituent IgG and IgM liquid calibrators that can be used in multiple analyte assay systems as positive controls to indicate the presence and the amount of one or more of the analytes of interest (e.g., IgG or IgM antibodies against certain pathogenic microorganisms) in a test sample. A calibrator in accordance with the present invention can be prepared by first obtaining a starting serum, which preferably contains at least one IgG or IgM antibody against a particular pathogenic microorganism. Heterologous IgG or IgM antibodies with desired specificity can then be isolated, purified, and dissolved in the serum. Such IgG or IgM antibodies are heterologous to the antibody or antibodies already present in the starting serum, but not necessary to each other. The present invention also resides in a method for using the calibrator.

All publications and U.S. Patents mentioned herein are hereby incorporated by reference.

II. Acquisition of Sera

Various methods known to those skilled in the art are suitable for obtaining a serum to use as a starting point for preparing a multiconstituent liquid IgG and IgM calibrator of this invention.

Generally, blood can be drawn from a subject, e.g., a human, following the standard practice of hospitals and clinics. An acellular preparation of the blood can be subsequently obtained in accordance with several established procedures. For example, serum as conventionally defined can be obtained following blood coagulation, and plasma as conventionally defined can be obtained following the removal of suspended particulate components. The term “serum” as used in this application includes both serum and plasma as conventionally defined. Preferably, a serum used as a starting point for preparing a liquid calibrator of this invention is from a subject known to have been exposed to at least one pathogen that is a desired test subject, such that the serum will already contain at least one IgG or IgM antibody that can serve as a positive control.

Upon acquiring a serum from a subject previously exposed to a particular pathogen and thereby expected to contain an antibody specifically reactive to the pathogen, an appropriate immunoassay, e.g., a Western blot assay, can be performed to confirm the presence of such antibody and, preferably, to estimate the level of such antibody in the serum. The description of the general format of immunoassays are provided below in detail.

III. Acquisition of Antibodies

Heterologous IgG or IgM antibodies against different antigens used to prepare liquid IgG and IgM calibrators of this invention can be obtained from a variety of sources. These antibodies may be naturally occurring antibodies that exist in different subjects and require isolation, purification, and preferably, quantification prior to their addition into a starting serum. These heterologous antibodies may also be artificial: they may be chimeric antibodies or antibodies recombinantly produced.

A. Naturally Occurring Antibodies

1. Production of Antibodies with Desired Specificity

Methods for producing polyclonal and monoclonal antibodies that react specifically with an immunogen of interest are known to those of skill in the art (see, e.g., Coligan, Current Protocols in Immunology Wiley/Greene, NY, 1991; Harlow and Lane, Antibodies: A Laboratory Manual Cold Spring Harbor Press, NY, 1989; Stites et al. (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y., 1986; and Kohler and Milstein Nature 256:495-497, 1975). Such techniques include antibody preparation by selection of antibodies from libraries of recombinant antibodies in phage or similar vectors (see, Huse et al., Science 246:1275-1281, 1989; and Ward et al., Nature 341:544-546, 1989).

In order to produce antisera containing antibodies with desired specificity for the construction of a liquid calibrator of this invention, the pathogenic organism of interest (e.g., a bacterium or a virus) or an antigenic fragment thereof (e.g., a purified or recombinant polypeptide from the bacterium or virus) can be used to immunize suitable animals, e.g., mice, rabbits, or primates. A standard adjuvant, such as Freund's adjuvant, can be used in accordance with a standard immunization protocol. Alternatively, a synthetic peptide derived from that particular pathogen can be conjugated to a carrier protein and subsequently used as an immunogen.

The animal's immune response to the immunogen preparation is monitored by taking test bleeds and determining the titer of reactivity to the antigen of interest. When appropriately high titers of antibody to the antigen are obtained, blood is collected from the animal and antisera are prepared. When appropriate, blood with high titers of desired antibodies may also be collected from a human subject with prior exposure to a particular infectious microorganism of interest. Further fractionation of the antisera to enrich antibodies specifically reactive to the antigen and purification of the antibodies can be accomplished subsequently, see, Harlow and Lane, supra, and general descriptions of antibody purification offered below.

Monoclonal antibodies may be obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519, 1976). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and the yield of the monoclonal antibodies produced by such cells may be enhanced by various techniques, including injection into the peritoneal cavity of a vertebrate host.

Additionally, monoclonal antibodies may also be recombinantly produced upon identification of nucleic acid sequences encoding an antibody with desired specificity or a binding fragment of such antibody by screening a human B cell cDNA library according to the general protocol outlined by Huse et al., supra. A more detailed description of antibody production by recombinant methods can be found in a later section.

2. Purification of Antibodies

Methods for antibody purification are well known in the field of biomedical research, some of which rely on the unique characteristics of the antibodies to be purified, whereas others are standard protein separation techniques suitable for a broad range of applications.

a. Solubility Fractionation

Salt fractionation can be used as an initial step to separate desired antibodies from other unwanted proteins. The preferred salt is ammonium sulfate, which precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The desired antibody is precipitated at an appropriate ammonium sulfate concentration according to its hydrophobicity and is then solubilized in a buffer with the excess salt removed if necessary, through either dialysis or diafiltration. Other methods that rely on solubility of proteins, such as cold ethanol precipitation, are well known to those of skill in the art and may also be used to prepare an antibody fraction from a protein mixture, such as a serum.

b. Size Differential Filtration

Based on a predicted molecular weight, an antibody can be isolated from proteins of greater and lesser sizes using ultrafiltration through membranes of different pore sizes (for example, Amicon or Millipore membranes). As a first step, a protein mixture (e.g., a serum or a cell culture supernatant) is ultrafiltered through a membrane with a pore size that has a lower molecular weight cut-off than the predicted molecular weight of the desired antibody. The retentate of the ultrafiltration is then ultrafiltered against a membrane with a molecular cut-off greater than the predicted molecular weight of the desired antibody. The antibody will pass through the membrane into the filtrate, which can then be processed in a next step of column chromatography.

c. Column Chromatography

The heterologous antibodies used for constructing the claimed liquid calibrator can also be separated from other proteins including other antibodies on the basis of their size, net surface charge, hydrophobicity, and affinity for ligands. Column chromatography is a frequently used method. For example, antibodies can be isolated from other non-antibody proteins using a column with immobilized protein A or protein G, which are bacterial cell wall proteins that bind to a domain in the Fc region of antibodies. Furthermore, antibodies against different antigens can be separated based on their distinct affinity to these antigens, which are immobilized to a column in a preferred format of column chromatography for antibody purification. All of these methods are well known in the art, and it will be apparent to one of skill that chromatographic techniques can be performed at any scale and using equipment from many different manufacturers (e.g., Pharmacia Biotech).

B. Artificially Produced Antibodies

1. General Approaches

Besides naturally-occurring antibodies, artificially produced antibodies may also be used to construct a multiconstituent liquid IgG and IgM calibrator of the present invention. The general methods for recombinantly producing antibodies with desired specificity are known to those skilled in the relevant art and are described in numerous publications. See, e.g., U.S. Pat. No. 5,665,570. Briefly, the genes encoding an antibody with desired specificity can be identified by screening a B cell cDNA library using various cloning techniques, e.g., a cloning method based on polymerase chain reaction (PCR), and subsequently expressed in suitable host cells. For a general description of recombinant DNA technology, see, e.g., Sambrook and Russell, Molecular Cloning: A Laboratory Manual 3d ed. 2001; Kriegler, Gene Transfer and Expression: A Laboratory Manual 1990; and Ausubel et al., Current Protocols in Molecular Biology 1994.

Another means for recombinantly producing antibodies with desired specificity relies on the chimeric antibody technology. Generally, the genes encoding the variable regions of a non-human monoclonal antibody (e.g., a murine antibody) are cloned and joined with the coding sequences for human constant regions to produce the so-called “humanized” antibodies. See, e.g., U.S. Pat. Nos. 5,502,167; 5,607,847; 5,773,247. Such humanized chimeric antibodies produced by host cells are suitable for constructing the claimed liquid IgG and IgM calibrators.

In addition, fully human antibodies against a specific antigen can be prepared by immunizing a transgenic animal that has been genetically manipulated so that its endogenous Ig loci has been inactivated and replaced with human Ig loci, to produce the antibodies in response to the antigenic challenge. Human monoclonal antibodies so produced are also suitable for practicing the present invention. Detailed description of this recently developed technology for producing human monoclonal antibodies of any desired specificity can be found in, e.g., U.S. Pat. Nos. 6,114,598; 6,150,584; 6,162,963. This approach differs from the first two in that it does not require expression of genes encoding an antibody with desired specificity in host cells; rather, filly human monoclonal antibodies can be obtained following the immunization procedure and antibody purification method outlined in the last section once a transgenic animal is established.

2. Host Cells

Various cell types, both prokaryotic and eukaryotic, are suitable for the expression of a recombinant antibody. These cell types include but are not limited to, for example, a variety of bacteria such as E. coli, Bacillus, and Salmonella, as well as eukaryotic cells such as yeast, insect cells, and mammalian cells. Suitable cells for gene expression are well known to those of skill in the art and are described in numerous scientific publications such as Sambrook and Russell, supra.

3. Expression Vectors

Upon acquisition of the nucleic acid sequences encoding a desired antibody, the sequences are typically cloned into an intermediate vector before transformation into prokaryotic or eukaryotic cells for replication and/or expression. The intermediate vector is typically a prokaryote vector such as a plasmid or shuttle vector.

To obtain high level expression of a cloned gene, such as the cDNA encoding an antibody with a desired specificity, one typically subclones the cDNA into an expression vector that contains a strong promoter to direct transcription, a transcription/translation terminator, and a ribosome binding site for translational initiation. Suitable bacterial promoters are well known in the art and fully described in scientific literature such as Sambrook and Russell, supra, and Ausubel et al, supra. Bacterial expression systems for expressing antibody chains of the recombinant catalytic polypeptide are available in, e.g., E. coli, Bacillus, and Salmonella (Palva et al., Gene, 22:229-235, 1983; Mosbach et al., Nature, 302:543-545, 1983). Kits for such expression systems are commercially available. Eukaryotic expression systems for mammalian cells, yeast, and insect cells are well known in the art and are also commercially available.

Selection of the promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function.

In addition to the promoter, the expression vector typically contains a transcription unit or expression cassette that contains all the additional elements required for the expression of the proteolytic antibody chain in host cells. A typical expression cassette thus contains a promoter operably linked to the nucleic acid sequence encoding the proteolytic antibody chain and signals required for efficient polyadenylation of the transcript, ribosome binding sites, and translation termination. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as MBP, GST, and LacZ. Epitope tags can also be added to recombinant proteins to provide convenient methods of isolation, e.g., c-myc or histidine tags.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a proteolytic antibody chain under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

4. Transfection Methods

Standard transfection methods are used to produce bacterial, mammalian, yeast, or insect cell lines that express large quantity of the desired recombinant antibody, which is then purified using standard techniques (see, e.g., Colley et al., J. Biol. Chem., 264:17619-17622, 1989; Guide to Protein Purification, in Methods in Enzymology, vol. 182 (Deutscher, ed.), 1990) and as described above. Transformation of eukaryotic and prokaryotic cells are performed according to standard techniques (see, e.g., Morrison, J. Bact., 132:349-351, 1977; Clark-Curtiss and Curtiss, Methods in Enzymology, 101:347-362 (Wu et al., eds), 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least both genes into the host cell capable of expressing the recombinant catalytic polypeptide.

After the expression vector is introduced into the cells, the transfected cells are cultured under conditions favoring expression of the antibody with desired specificity, which is screened for (e.g., using hybridization assays and electrophoresis) and recovered from the culture using standard techniques identified below.

5. Purification of Recombinant Antibodies

The recombinant antibodies may be purified to substantial purity by standard techniques as described above, including selective precipitation with such substances as ammonium sulfate; column chromatography, gel filtration, immunopurification methods, and others (see, e.g., U.S. Pat. No. 4,673,641; Scopes, Protein Purification: Principles and Practice, 1982; Sambrook and Russell, supra; and Ausubel et al., supra).

IV. Assembly of Multiconstituent Liquid Calibrator

Upon acquisition of all necessary IgG and IgM antibodies from various sources, the heterologous antibodies are quantified according to standard methods for protein quantification, e.g., Bradford method, and subsequently dissolved in the starting serum to produce the multiconstituent IgG and IgM liquid calibrator of the present invention. Calibrators of the present invention should be made to cover a range of approximately 0.5 to 4.0 times of the cutoff value with regard to each antibody tested for. The cutoff value is a predetermined level above which a signal would indicate a current or past infection in a test subject.

There are at least two heterologous antibodies present in the liquid IgG and IgM calibrators of this invention. Preferably, the calibrators include at least three, four, five, six, seven, eight, nine, ten, eleven, twelve, thirteen, fourteen, fifteen, sixteen, seventeen, or even more antibodies, which are independently IgG or IgM. Every antibody, however, need not be heterologous to every other antibody that may also be present in the same calibrator. The heterologous antibodies need not be dissolved in the serum in any particular order.

V. Detection of Antibody-Antigen Complex

Various formats of immunoassays are known to those skilled in the art to qualitatively and quantitatively detect the presence of an antibody in a complex with a predetermined antigen. Biological samples from a subject (e.g., a human patient suspected to have been infected with one or more of a list of predetermined pathogenic organisms), such as blood preparations or tissue samples, can be used in the immunoassays to detected the presence and the level of antibody or antibodies with specified reactivity, depending on the infection by particular pathogen(s) to be tested. For a review of immunological and immunoassay procedures in general, see, e.g., Harlow and Lane, supra; Stites, supra; U.S. Pat. Nos. 4,366,241; 4,376,110; 4,517,288; and 4,837,168.

A. Labeling in Immunoassays

Immunoassays often utilize a labeling agent to specifically bind to and label the binding complex formed by an antibody and its specific antigen. The labeling agent may itself be one of the moieties comprising the antibody/target protein complex, or may be a third moiety, such as another antibody, that specifically binds to the antibody/antigen complex. A label may be detectable by spectroscopic, photochemical, biochemical, immunochemical, electrical, optical, or chemical means. Some examples are, but not limited to, magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²p), enzymes (e.g., horse radish peroxidase, alkaline phosphatase, and others commonly used in an ELISA), and colorimetric labels such as colloidal gold or colored glass or plastic (e.g., polystyrene, polypropylene, latex, etc.) beads.

In some cases, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al., J. Immunol., 111:1401-1406, 1973; and Akerstrom, et al., J. Immunol., 135:2589-2542, 1985).

B. Immunoassay Formats

Immunoassays for detecting antibody or antibodies against specific antigens from patient samples may be either competitive or noncompetitive. Noncompetitive immunoassays are assays in which the amount of captured antibody is directly measured. In a preferred “sandwich” assay, for example, the antigen of a predetermined pathogen can be bound directly to a solid substrate where the antigen is immobilized. It then captures the antibody specifically reactive to it in test samples. The antigen/antibody complex thus immobilized is then bound by a labeling agent, such as a second antibody bearing a label. Alternatively, the second antibody may lack a label, but it may, in turn, be bound by a labeled third antibody specific to antibodies of the species from which the second antibody is derived. The second antibody can also be modified with a detectable moiety, such as biotin, to which a third labeled molecule can specifically bind, such as enzyme-labeled streptavidin.

In competitive assays, the amount of antibody of interest in a sample is measured indirectly by measuring the amount of an added (exogenous) antibody with same specificity displaced (or competed away) from the antigen specifically recognized by the antibody present in the sample. In a typical example of such an assay, the antigen is immobilized and the exogenous antibody is labeled. Since the amount of the exogenous antibody bound to the antigen is inversely proportional to the concentration of the antibody present in the sample, the antibody level in the sample can thus be determined based on the amount of exogenous antibody bound to the antigen and thus immobilized.

The claimed multiconstituent IgG and IgM liquid calibrators may also be used in other assay formats such as western blot (immunoblot) analysis and liposome immunoassays (LIA, see, Monroe et al., Amer. Clin. Prod. Rev., 5:34-41, 1986).

Flow Cytometry

Flow cytometry is one of the preferred methods for detecting the presence of multiple antibodies in a biological sample according to the present invention. Each of multiple antigens specific for the multiple antibodies is conjugated to a distinct label, such as a fluorescent molecule, so that antigen-antibody complexes containing different antigens can be distinguished. Methods of and instrumentation for flow cytometry are known in the art, and can be used in the practice of the present invention. Flow cytometry in general resides in the passage of a suspension of the microparticles as a stream past a light beam and electro-optical sensors, in such a manner that only one particle at a time passes the region of the sensors. As each particle passes this region, the light beam is perturbed by the presence of the particle, and the resulting scattered and fluoresced light is detected. The optical signals are used by the instrumentation to identify the subgroup to which each particle belongs, along with the presence and amount of label, so that individual assay results are achieved. Detailed descriptions of instrumentation and methods for flow cytometry are found in the literature. Examples are McHugh, “Flow Microsphere Immunoassay for the Quantitative and Simultaneous Detection of Multiple Soluble Analytes,” Methods in Cell Biology 42, Part B (Academic Press, 1994); McHugh et al., “Microsphere-Based Fluorescence Immunoassays Using Flow Cytometry Instrumentation,” Clinical Flow Cytometry, Bauer, K. D., et al., eds. (Baltimore, Md., USA: Williams and Williams, 1993), pp. 535-544; Lindmo et al., “Immmunometric Assay Using Mixtures of Two Particle Types of Different Affinity,” J. Immunol. Meth. 126:183-189, 1990; McHugh, “Flow Cytometry and the Application of Microsphere-Based Fluorescence Immunoassays,” Immunochemica 5:116, 1991; Horan et al., “Fluid Phase Particle Fluorescence Analysis: Rheumatoid Factor Specificity Evaluated by Laser Flow Cytophotometry,” Immunoassays in the Clinical Laboratory, 185-189 (Liss 1979); Wilson et al., “A New Microsphere-Based Immunofluorescence Assay Using Flow Cytometry,” J. Immunol. Meth. 107: 225-230, 1988; Fulwyler et al., “Flow Microsphere Immunoassay for the Quantitative and Simultaneous Detection of Multiple Soluble Analytes,” Meth. Cell Biol. 33: 613-629, 1990; Coulter Electronics Inc., United Kingdom Patent No. 1,561,042 (published Feb. 13, 1980); and Steinkamp et al., Review of Scientific Instruments 44(9): 1301-1310, 1973.

The particles used in the practice of this invention are preferably microscopic in size and formed of a polymeric material. Polymers that will be useful as microparticles are those that are chemically inert relative to the components of the biological sample and to the assay reagents other than the binding member coatings that are affixed to the microparticle surface. Suitable microparticle materials will also have minimal autofluorescence, will be solid and insoluble in the sample and in any buffers, solvents, carriers, diluents, or suspending agents used in the assay, and will be capable of affixing to the appropriate coating material, preferably through covalent bonding. Examples of suitable polymers are polyesters, polyethers, polyolefins, polyalkylene oxides, polyamides, polyurethanes, polysaccharides, celluloses, and polyisoprenes. Crosslinking is useful in many polymers for imparting structural integrity and rigidity to the microparticle. The size range of the microparticles can vary and particular size ranges are not critical to the invention. In most cases, the microparticles will range in diameter from about 0.3 micrometers to about 100 micrometers, and preferably from about 0.5 micrometers to about 40 micrometers.

To facilitate the particle recovery and washing steps of the assay, the particles preferably contain a magnetically responsive material, i.e., any material that responds to a magnetic field. Separation of the solid and liquid phases, either after incubation or after a washing step, is then achieved by imposing a magnetic field on the reaction vessel in which the suspension is incubated, causing the particles to adhere to the wall of the vessel and thereby permitting the liquid to be removed by decantation or aspiration. Magnetically responsive materials of interest in this invention include paramagnetic materials, ferromagnetic materials, ferrimagnetic materials, and metamagnetic materials. Paramagnetic materials are preferred. Examples are iron, nickel, and cobalt, as well as metal oxides such as Fe₃O₄, BaFe₁₂O₁₉, CoO, NiO, Mn₂O₃, Cr₂O₃, and CoMnP.

The magnetically responsive material can be dispersed throughout the polymer, applied as a coating on the polymer surface or as one of two or more coatings on the surface, or incorporated or affixed in any other manner that secures the material in to the particle. The quantity of magnetically responsive material in the particle is not critical and can vary over a wide range. The quantity can affect the density of the microparticle, however, and both the quantity and the particle size can affect the ease of maintaining the microparticle in suspension for purposes of achieving maximal contact between the liquid and solid phase and for facilitating flow cytometry. An excessive quantity of magnetically responsive material in the microparticles may produce autofluorescence at a level high enough to interfere with the assay results. It is therefore preferred that the concentration of magnetically responsive material be low enough to minimize any autofluorescence emanating from the material. With these considerations in mind, the magnetically responsive material in a particle in accordance with this invention preferably ranges from about 0.05% to about 75% by weight of the particle as a whole. A more preferred weight percent range is from about 1% to about 50%, a still more preferred weight percent range is from about 2% to about 25%, and an even more preferred weight percent range is from about 2% to about 8%.

Coating of the particle surface with the appropriate assay reagent can be achieved by electrostatic attraction, specific affinity interaction, hydrophobic interaction, or covalent bonding. Covalent bonding is preferred. The polymer can be derivatized with functional groups for covalent attachment of the assay reagent by conventional means, notably by the use of monomers that contain the functional groups, such monomers serving either as the sole monomer or as a co-monomer. Examples of suitable functional groups are amine groups (—NH₂), ammonium groups (—NH₃⁺ or —NR₃⁺), hydroxyl groups (—OH), carboxylic acid groups (—COOH), and isocyanate groups (—NCO). Useful monomers for introducing carboxylic acid groups into polyolefins, for example, are acrylic acid and methacrylic acid.

Linkers can be used as a means of increasing the density of antibody-recognizable eitopes on the particle surface and decreasing steric hindrance. This will increase the range and sensitivity of the assay. Linkers can also be used as a means of adding specific types of reactive groups to the solid phase surface if needed to secure the particular coating materials of this invention. Examples of suitable useful functional groups are polylysine, polyaspartic acid, polyglutamic acid, and polyarginine.

In general, care should be taken to avoid the use of particles that exhibit high autofluorescence. Particles formed by conventional emulsion polymerization techniques from a wide variety of starting monomers are generally suitable since they exhibit at most a low level of autofluorescence. Conversely, particles that have been modified to increase their porosity and hence their surface area, i.e., those particles that are referred to in the literature as “macroporous” particles, are less desirable since they tend to exhibit high autofluorescence. A further consideration is that autofluorescence increases with increasing size and increasing percentage of divinylbenzene monomer.

The labels used in the labeled binding members may be any label that is capable of emitting detectable signal. Preferred such labels are fluorophores. A vast array of fluorophores are reported in the literature and thus known to those skilled in the art, and many are readily available from commercial suppliers to the biotechnology industry. Literature sources for fluorophores include Cardullo et al., Proc. Natl. Acad. Sci. USA 85: 8790-8794, 1988; Dexter, D. L., J. of Chemical Physics 21: 836-850, 1953; Hochstrasser et al., Biophysical Chemistry 45: 133-141, 1992; Selvin, P., Methods in Enzymology 246: 300-334, 1995; Steinberg, I. Ann. Rev. Biochem., 40: 83-114, 1971; Stryer, L. Ann. Rev. Biochem., 47: 819-846 (1978); Wang et al., Tetrahedron Letters 31: 6493-6496 (1990); Wang et al., Anal. Chem. 67: 1197-1203, 1995.

The following is a list of examples of fluorophores:
4-acetamido-4′-isothiocyanatostilbene-2,2′disulfonic acid
acridine
acridine isothiocyanate
5-(2′-aminoethyl)aminonaphthalene-1-sulfonic acid (EDANS)
4-amino-N-[3-vinylsulfonyl)phenyl]naphthalimide-3,5 disulfonate
N-(4-anilino-1-naphthyl)maleimide
anthranilamide
BODIPY
Brilliant Yellow
coumarin
7-amino-4-methylcoumarin (AMC, Coumarin 120)
7-amino-4-trifluoromethylcoumarin (Coumarin 151)
cyanine dyes
cyanosine
4′,6-diaminidino-2-phenylindole (DAPI)
5′,5″-dibromopyrogallol-sulfonaphthalein (Bromopyrogallol Red)
7-diethylamino-3-(4′-isothiocyanatophenyl)-4-methylcoumarin diethylenetriamine pentaacetate
4,4′-diisothiocyanatodihydro-stilbene-2,2′-disulfonic acid
4,4′-diisothiocyanatostilbene-2,2′-disulfonic acid
5-[dimethylamino]naphthalene-1-sulfonyl chloride (DNS, dansylchloride)
4-(4′-dimethylaminophenylazo) benzoic acid (DABCYL)
4-dimethylaminophenylazophenyl-4′-isothiocyanate (DABITC)
eosin
eosin isothiocyanate
erythrosin B
erythrosin isothiocyanate
ethidium
5-carboxyfluorescein (FAM)
5-(4,6-dichlorotriazin-2-yl)aminofluorescein (DTAF)
2′,7′-dimethoxy-4′5′-dichloro-6-carboxyfluorescein (JOE)
fluorescein
fluorescein isothiocyanate
fluorescamine
IR144
IR1446
Malachite Green isothiocyanate
4-methylumbelliferone
ortho cresolphthalein
nitrotyrosine
pararosaniline
Phenol Red
B-phycoerythrin
o-phthaldialdehyde
pyrene
pyrene butyrate
succinimidyl 1-pyrene butyrate
quantum dots
Reactive Red 4 (Cibacron™ Brilliant Red 3B-A)
6-carboxy-X-rhodamine (ROX)
6-carboxyrhodamine (R6G)
lissamine rhodamine B sulfonyl chloride rhodamine (Rhod)
rhodamine B
rhodamine 123
rhodamine X isothiocyanate
sulforhodamine B
sulforhodamine 101
sulfonyl chloride derivative of sulforhodamine 101 (Texas Red)
N,N,N′,N′-tetramethyl-6-carboxyrhodaminne (TAMRA)
tetramethyl rhodamine
tetramethyl rhodamine isothiocyanate (TRITC)
riboflavin
rosolic acid
lanthamide chelate derivatives

The attachment of any of these fluorophores to the binding molecules described above to form assay reagents for use in the practice of this invention is achieved by conventional covalent bonding, using appropriate functional groups on the fluorophores and on the binding members. The recognition of such groups and the reactions to form the linkages will be readily apparent to those skilled in the art.

Similarly, methods of and instrumentation for applying and removing a magnetic field as part of an automated assay are known to those skilled in the art and reported in the literature. Examples of literature reports are Forrest et al., U.S. Pat. No. 4,141,687 (Technicon Instruments Corporation, Feb. 27, 1979); Ithakissios, U.S. Pat. No. 4,115,534 (Minnesota Mining and Manufacturing Company, Sep. 19, 1978); Vlieger, A. M., et al., Analytical Biochemistry 205:1-7, 1992; Dudley, Journal of Clinical Immunoassay 14:77-82, 1991; and Smart, Journal of Clinical Immunoassay 15:246-251, 1992. All of the citations in this and the preceding paragraph are incorporated herein by reference.

### EXAMPLES

The following examples are for the purposes of illustration and not limitation.

### Example 1

A multiconstituent IgG and IgM liquid calibrator was constructed. It contained the antibodies against Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

### Example 2

A multiconstituent IgG and IgM liquid calibrator was constructed. It contained the antibodies against Rubella virus, Mumps Virus, and Measles Virus.

### Example 3

A multiconstituent IgG and IgM liquid calibrator was constructed. It contained the antibodies against Toxoplasma gondii, Rubella virus, Cytomegalovirus (CMV), Herpes Simplex Virus type-1 (HSV-1), and Herpes Simplex Virus type-2 (HSV-2).

### Example 4

A multiconstituent IgG and IgM liquid calibrator was constructed. It contained the antibodies against antigens derived from Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Varicella Zoster Virus, Treponema pallidum, Helicobacter pylori, Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

### Example 5

A multiconstituent IgG and IgM liquid calibrator is constructed. It contains the antibodies against the following organisms and specific antigens of Epstein-Barr Virus (EBV): Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Epstein-Barr Virus Viral Capsid Antigen (EBV-VCA), Epstein-Barr Virus Nuclear Antigen type-1 (EBV-NA1), Epstein-Barr Virus Early Antigen Diffused (EBV-EAD), Varicella Zoster Virus, Borrelia burgdorferi, Treponema pallidum, Helicobacter pylori, and Mycoplasma pneumoniae.

1. A composition comprising a serum in which are dissolved a plurality of heterologous antibodies that are independently IgG or IgM, each of said antibodies specifically binding to different antigens.

2. The composition of claim 1, wherein said plurality of heterologous antibodies comprises three said heterologous antibodies.

3. The composition of claim 1, wherein said plurality of heterologous antibodies comprises five said heterologous antibodies.

4. The composition of claim 1, wherein said plurality of heterologous antibodies comprises ten said heterologous antibodies.

5. The composition of claim 1, wherein said plurality of heterologous antibodies comprises fifteen said heterologous antibodies.

6. The composition of claim 1, wherein said different antigens are derived from one or more organisms independently selected from the group consisting of Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Epstein-Barr Virus (EBV), Varicella Zoster Virus, Borrelia burgdorferi, Treponema pallidum, Helicobacter pylori, and Mycoplasma pneumoniae.

7. The composition of claim 6, wherein said different antigens derived from Epstein-Barr Virus (EBV) comprise antigens derived from Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

8. The composition of claim 6, wherein said different antigens are derived from Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

9. The composition of claim 6, wherein said different antigens are derived from Rubella virus, Mumps Virus, and Measles Virus.

10. The composition of claim 6, wherein said different antigens are derived from Toxoplasma gondii, Rubella virus, Cytomegalovirus (CMV), Herpes Simplex Virus type-1 (HSV-1), and Herpes Simplex Virus type-2 (HSV-2).

11. The composition of claim 6, wherein said different antigens are derived from Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Varicella Zoster Virus, Treponema pallidum, Helicobacter pylori, Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

12. The composition of claim 6, wherein said different antigens are derived from Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Varicella Zoster Virus, Borrelia burgdorferi, Treponema pallidum, Helicobacter pylori, Mycoplasmapneumoniae, Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

13. A method for analyzing a biological sample to detect the presence and amount of IgG or IgM antibodies to a plurality of predetermined different antigens, said method comprising:
(a) contacting said biological sample with said plurality of predetermined different antigens under conditions sufficient to allow the formation of antigen/antibody complexes between any of said plurality of predetermined different antigens and any of IgG or IgM antibodies present in said sample that specifically bind to any of said plurality of predetermined different antigens;
(b) detecting any antigen/antibody complexes thus formed; and
(c) comparing the result of step (b) to a result obtained by
(i) contacting a control composition with said plurality of predetermined different antigens, said control composition comprising a serum in which are dissolved a plurality of heterologous antibodies that are independently IgG or IgM, each of said antibodies specifically binding to one of said plurality of predetermined different antigens, and
(ii) detecting the formation of antigen/antibody complexes between any of said plurality of predetermined different antigens and any of said plurality of heterologous antibodies present in said control composition,
to identify any of said IgG or IgM antibodies present in said biological sample and to quantify the levels in said biological sample of any of said IgG or IgM antibodies thus identified.

14. The method of claim 13, wherein said plurality of heterologous antibodies comprises three said heterologous antibodies.

15. The method of claim 13, wherein said plurality of heterologous antibodies comprises five said heterologous antibodies.

16. The method of claim 13, wherein said plurality of heterologous antibodies comprises ten said heterologous antibodies.

17. The method of claim 13, wherein said plurality of heterologous antibodies comprises fifteen said heterologous antibodies.

18. The method of claim 13, wherein said different antigens are derived from one or more organisms independently selected from the group consisting of Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Epstein-Barr Virus (EBV), Varicella Zoster Virus, Borrelia burgdorferi, Treponema pallidum, Helicobacter pylori, and Mycoplasma pneumoniae.

19. The method of claim 18, wherein said different antigens derived from Epstein-Barr Virus (EBV) comprise antigens derived from Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

20. The method of claim 18, wherein said different antigens are derived from Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

21. The method of claim 18, wherein said different antigens are derived from Rubella virus, Mumps Virus, and Measles Virus.

22. The method of claim 18, wherein said different antigens are derived from Toxoplasma gondii, Rubella virus, Cytomegalovirus (CMV), Herpes Simplex Virus type-1 (HSV-1), and Herpes Simplex Virus type-2 (HSV-2).

23. The method of claim 18, wherein said different antigens are derived from Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Varicella Zoster Virus, Treponema pallidum, Helicobacter pylori, Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

24. The method of claim 18, wherein said different antigens are derived from Toxoplasma gondii, Rubella virus, Cytomegalovirus (CMV), Herpes Simplex Virus type-1 (HSV-1), Herpes Simplex Virus type-2 (HSV-2), Mumps Virus, Measles Virus, Varicella Zoster Virus, Borrelia burgdorferi, Treponema pallidum, Helicobacter pylori, and Mycoplasma pneumoniae, Epstein-Barr Virus Viral Capsid Antigen (EBV-VCA), Epstein-Barr Virus Nuclear Antigen type-1 (EBV-NA1), and Epstein-Barr Virus Early Antigen Diffused (EBV-EAD).

25. The method of claim 13, wherein steps (b) and (c)(ii) are performed by flow cytometry.
